(19) United States
(12) Patent Application Publication
(10) Pub. No.: US 2024/0042108 A1
(43) Pub. Date: Feb. 8, 2024

(54) Orthopedic Internal Fixation Implanted Medical Device

(71) Applicant: BIOTYX MEDICAL (SHENZHEN) CO., LTD., SHENZHEN, GUANGDONG (CN)

(72) Inventors: Deyuan Zhang, Shenzhen (CN); Haiping Qi, Shenzhen (CN)

(73) Assignee: BIOTYX MEDICAL (SHENZHEN) CO., LTD., SHENZHEN, GUANGDONG (CN)

(21) Appl. No.: 18/267,708

(22) PCT Filed: Dec. 28, 2021
(86) PCT No.: PCT/CN2021/141841

§ 371 Date: Jun. 15, 2023

(30) Foreign Application Priority Data
Dec. 28, 2020 (CN) 202011606661.0

(51) Int. Cl. A61L 31/12; A61L 31/02; A61L 31/14

(52) U.S. Cl. CPC A61L 31/124; A61L 31/022; A61L 31/148

(57) ABSTRACT

The present invention relates to the technical field of medical devices, and in particular to an orthopedic internal fixation implant medical device, including an iron matrix and a filling material including polylactic acid and an alkaline substance. The polylactic acid has a weight-average molecular weight of M_{w} kDa, the alkaline substance includes a metal element, the mass ratio of the metal element in the alkaline substance to the polylactic acid is p, and the p and the M_{w} satisfy a formula of 2M_{w}\hat{ }−0.8≤p≤30M_{w}\hat{ }−0.5. The orthopedic internal fixation implant medical device, with good mechanical properties, can control local pH values and induce bone healing.

### TECHNICAL FIELD

The present invention relates to the technical field of medical devices, and in particular to an orthopedic internal fixation implant medical device.

### BACKGROUND ART

Traditional orthopedic internal fixation devices are generally made of permanent metals such as stainless steel, titanium-based alloy, and cobalt-based alloy. These materials have excellent mechanical properties and biocompatibility, but they may have problems such as erosion, allergy, and osteoporosis due to the stress shielding effect after long-term retention in the human body. This requires a second surgery to remove the fracture after the patient has healed, which greatly increases the patient's pain and economic burden. Therefore, absorbable orthopedic fixtures made of degradable biomedical materials have been extensively studied for clinical use in recent years. The most attractive advantages of absorbable orthopedic internal fixation materials over permanent metal internal fixtures are that they do not require subsequent surgical removal, greatly reducing the patient's pain.

Currently, absorbable orthopedic fixation materials mainly include absorbable polymers, magnesium, and alloys thereof.

Magnesium alloy orthopedic internal fixation materials, with good biocompatibility, can be degraded in vivo to avoid the pain of subsequent surgical removal. Moreover, magnesium ions released by the materials can also promote the proliferation and differentiation of bone cells and promote bone growth and healing. The elastic modulus of magnesium alloy is close to that of human bone, which can effectively reduce the stress shielding effect. Meanwhile, the mechanical properties, such as tensile strength, of magnesium alloy are much higher than that of degradable polymer materials in clinical application, which can better meet clinical needs. However, the mechanical properties of magnesium-based alloy still do not reach the level of permanent metal implant materials, so the range of clinical application is limited. Magnesium-based alloys are still not optimal for use in the load-bearing parts, and can only be used in the non-load-bearing and less active positions. Second, magnesium-based alloys, with a faster degradation rate, will lose effective supporting and fixing function prematurely in response to implantation into the medical device. During the degradation, it will increase the local pH of the implantation site and generate excessive hydrogen bubbles, which are not conducive to the healing of the bone injury site.

Absorbable polymers, such as polylactic acid and polycaprolactone, have good biocompatibility, and a lot of clinical data have been accumulated. Polylactic acid with high molecular weight is melted and processed into a shape, so as to make an orthopedic internal fixation implant medical device with certain mechanical strength. Compared with the traditional permanent metal materials, the disadvantages of absorbable polymers are as follows: (1) Due to poor osteoconductivity and the slow rate of repair of bone defects, it is difficult to achieve complete bone repair for larger bone defects; (2) Due to poor mechanical properties and insufficient mechanical strength, they are generally not applicable to the bearing parts (such as limbs), and only applicable to the fixation of cancellous bone, joint bone or less active bone in various non-bearing parts; (3) Due to fast early degradation rate, they cannot meet the mechanical property requirements before new bone tissue grows out; (4) The degradation of polylactic acid will generate an acidic environment, which will easily lead to serious inflammatory reactions at the implantation site. These drawbacks have greatly limited the application of absorbable polymer-based internal fixation implant medical devices. There is still room for improvement in the local slightly acidic environment generated by polylactic acid and in the osteoinductive capacity.

### SUMMARY OF THE INVENTION

An object of the present invention is to provide an orthopedic internal fixation implant medical device with better mechanical properties, better control of local pH values, reduction of local inflammatory reactions, controllable degradation rate, and induction of bone healing.

According to a first aspect of the present invention, there is provided an orthopedic internal fixation implant medical device including an iron matrix and a filling material, the filling material including polylactic acid and an alkaline substance, where the polylactic acid has a weight-average molecular weight of M_{w} kDa, the alkaline substance includes a metal element, the mass ratio of the metal element in the alkaline substance to the polylactic acid is p, and the p and the M_{w} satisfy a formula of 2M_{w}\hat{ }−0.8≤p≤30M_{w}\hat{ }−0.5.

The iron matrix in the above orthopedic internal fixation implant medical device can provide sufficient mechanical support, which solves the problem of insufficient mechanical properties of polylactic acid orthopedic internal fixation medical devices and magnesium alloy orthopedic internal fixation medical devices. The filling material includes polylactic acid and an alkaline substance, where the alkaline substance can neutralize the acidity of the degradation product of the polylactic acid in the early stage, and the mass ratio of the metal element in the alkaline substance to the polylactic acid is set as p, the polylactic acid has a molecular weight of M_{w} kDa, and the p and the M_{w} satisfy a formula of 2M_{w}\hat{ }−0.8≤p≤30M_{w}\hat{ }−0.5. In response to satisfying the formula, it can not only keep the local pH stable and make the pH neutral to reduce the inflammatory reactions, which is beneficial to bone repair, but can also slow down the early degradation rate of the iron matrix to maintain good mechanical properties during bone healing.

In one embodiment, the polylactic acid has a weight-average molecular weight of 5 kDa to 1000 kDa.

In one embodiment, the alkaline substance is selected from one or more of magnesium, magnesium alloy, zinc, zinc alloy, magnesium oxide, magnesium hydroxide, zinc oxide, zinc hydroxide, magnesium carbonate, zinc carbonate, magnesium phosphate, zinc phosphate, sodium carbonate, sodium bicarbonate, calcium oxide, calcium hydroxide, calcium carbonate, calcium phosphate, and hydroxyapatite.

In one embodiment, the alkaline substance is one or more of a powder, granule, block or rod.

In one embodiment, the alkaline substance is a combination of hydroxyapatite and at least one of magnesium, magnesium alloy, zinc, zinc alloy, magnesium oxide, magnesium hydroxide, zinc oxide, zinc hydroxide, magnesium carbonate, zinc carbonate, magnesium phosphate, zinc phosphate, sodium carbonate, sodium bicarbonate, calcium oxide, calcium hydroxide, calcium carbonate, and calcium phosphate. That is, the alkaline substance includes hydroxyapatite, and also includes at least one of magnesium, magnesium alloy, zinc, zinc alloy, magnesium oxide, magnesium hydroxide, zinc oxide, zinc hydroxide, magnesium carbonate, zinc carbonate, magnesium phosphate, zinc phosphate, sodium carbonate, sodium bicarbonate, calcium oxide, calcium hydroxide, calcium carbonate, and calcium phosphate.

In one embodiment, a mass of the hydroxyapatite is 1% to 10% of that of the orthopedic internal fixation medical device.

In one embodiment, the polylactic acid is poly-dl-lactic acid or poly-L-lactic acid.

In one embodiment, the iron matrix is a hollow structure, and the filling material is filled inside the iron matrix; or the iron matrix is a mesh skeleton structure, and the filling material is filled in meshes of the mesh skeleton structure; or the iron matrix is a hollow space skeleton structure, and the filling material is filled inside the hollow space of the iron matrix; or a groove or hole is disposed on a surface of the iron matrix, and the filling material is filled in the groove or hole of the iron matrix; or the filling material is coated on the surface of the iron matrix.

In one embodiment, the orthopedic internal fixation implant medical device is a bone nail, bone plate, bone rod, or bone mesh.

In one embodiment, the iron matrix is pure iron, low alloy steel, or iron-based alloy with a carbon content of not more than 2.5 wt. %.

### BRIEF DESCRIPTION OF THE DRAWINGS

Various other advantages and benefits will become apparent to those of ordinary skill in the art upon reading the following detailed description of the preferred implementations. Accompanying drawings are only for the purposes of illustrating the preferred implementations and are not to be construed as limiting the present invention. And throughout the accompanying drawings, the same components are represented by the same reference numerals. In which:

FIG. 1 is a cross-section view of an orthopedic internal fixation implant medical device provided in Embodiment 1.

FIG. 2 is a cross-section view of an orthopedic internal fixation implant medical device provided in Embodiment 3.

FIG. 3 is a cross-section view of an orthopedic internal fixation implant medical device provided in Embodiment 5.

FIG. 4 is a cross-section view of an orthopedic internal fixation implant medical device provided in Embodiment 7.

FIG. 5 is a cross-section view of an orthopedic internal fixation implant medical device provided in Embodiment 9.

FIG. 6 is a cross-section view of an orthopedic internal fixation implant medical device provided in Embodiment 10.

### DETAILED DESCRIPTION OF THE INVENTION

Exemplary implementations of the present invention will be described in more detail below with reference to the accompanying drawings. While exemplary implementations of the present invention have been illustrated in the accompanying drawings, it is to be understood that the present invention may be embodied in various forms and should not be construed as limited to the implementations set forth herein. Instead, these embodiments are provided to enable a more thorough understanding of the present invention and to fully convey the scope of the present invention to those skilled in the art.

It is to be understood that the terms used herein are for the purpose of describing particular example implementations only and are not intended to be limiting. As used herein, singular forms “a”, “an”, and “the” are intended to include plural forms as well, unless the context clearly indicates otherwise. Terms “include”, “comprise”, “contain”, and “have” are inclusive and therefore specify the presence of stated features, steps, operations, elements, and/or components, but do not preclude the presence or addition of one or more other features, steps, operations, elements, components, and/or combination thereof. The method steps, processes, and operations described herein are not to be construed as necessarily requiring their performance in the particular order described or illustrated, unless an order of performance is expressly stated. It should also be understood that additional or alternative steps may be used.

The embodiment provides an orthopedic internal fixation implant medical device, including an iron matrix and a filling material including polylactic acid and an alkaline substance, where the polylactic acid has a weight-average molecular weight of M_{w} kDa, the alkaline substance includes a metal element, the mass ratio of the metal element in the alkaline substance to the polylactic acid is p, and the p and the M_{w} satisfy a formula of 2M_{w}\hat{ }−0.8≤p≤30M_{w}\hat{ }−0.5. The metal element may include a metal cation or a metal atom. The formula is written as that the mass ratio p of the metal element in the alkaline substance to the polylactic acid is greater than or equal to 2 times the −0.8 power of the weight-average molecular weight M_{w} of the polylactic acid, and the mass ratio p of the metal element in the alkaline substance to the polylactic acid is less than or equal to 30 times the −0.5 power of the weight-average molecular weight M_{w} of the polylactic acid.

The iron matrix can provide sufficient mechanical support to solve the problem of insufficient mechanical properties of polylactic acid orthopedic internal fixation medical devices and magnesium alloy orthopedic internal fixation medical devices. The alkaline substance can neutralize the acidity of the degradation product of the polylactic acid in the early stage. In response to p and M_{w} satisfying the formula, it can not only keep the local pH stable and make the pH neutral to reduce the inflammatory reactions, which is beneficial to bone repair, but can also slow down the early degradation rate of the iron matrix to maintain good mechanical properties during bone healing.

In one embodiment, the polylactic acid has a weight-average molecular weight of 5 kDa to 1000 kDa. Preferably, the polylactic acid has a weight-average molecular weight of 100 kDa to 500 kDa, so that the early acidity of the polylactic acid is weaker, and at the same time, the degradation cycle becomes longer, which is beneficial to the accelerated degradation of the iron matrix in the later stage.

In one embodiment, the alkaline substance is selected from one or more of magnesium, magnesium alloy, zinc, zinc alloy, magnesium oxide, magnesium hydroxide, zinc oxide, zinc hydroxide, magnesium carbonate, zinc carbonate, magnesium phosphate, zinc phosphate, sodium carbonate, sodium bicarbonate, calcium oxide, calcium hydroxide, calcium carbonate, calcium phosphate, and hydroxyapatite. Preferably, the alkaline substance is selected from one or more of magnesium oxide, magnesium hydroxide, zinc oxide, zinc hydroxide, magnesium carbonate, zinc carbonate, magnesium phosphate, zinc phosphate, sodium carbonate, sodium bicarbonate, calcium oxide, calcium hydroxide, calcium carbonate, calcium phosphate, and hydroxyapatite. Among them, the above oxides or hydroxides, or weak acid and strong base salts can avoid the hydrogen bubbles formed by the reaction of such metals as magnesium, magnesium alloy, zinc, and zinc alloy with the polylactic acid, which is conducive to tissue growth and repair. At the same time, the alkaline substance, with lower alkalinity than that of oxides or hydroxides, has better biocompatibility. Preferably, the alkaline substance is a combination of hydroxyapatite and at least one of magnesium oxide, magnesium hydroxide, zinc oxide, zinc hydroxide, magnesium carbonate, zinc carbonate, magnesium phosphate, zinc phosphate, sodium carbonate, sodium bicarbonate, calcium oxide, calcium hydroxide, calcium carbonate, and calcium phosphate, where the hydroxyapatite is used for enhancing bioactivity and promoting bone healing.

In one embodiment, the alkaline substance is one or more of a powder, granule, block, or rod, which facilitates the addition of the alkaline substance to the filling material in various states.

In one embodiment, the alkaline substance includes hydroxyapatite, the mass of which is 1% to 10% of that of the orthopedic internal fixation medical device.

In one embodiment, the polylactic acid is poly-dl-lactic acid or poly-L-lactic acid.

In one embodiment, the iron matrix is pure iron, low alloy steel, or iron-based alloy with a carbon content of not more than 2.5 wt. %. The low alloy steel is an alloy steel with a total amount of alloying elements of less than 5%. Preferably, the iron matrix is nitrided iron with a carbon content of less than or equal to 0.25%, belonging to an iron-based alloy with a carbon content of not more than 2.5 wt. %. Nitrided iron has better mechanical properties.

In the embodiment of the present invention, the connection relationship between the iron matrix and the filling material has various forms. The iron matrix is a hollow structure, and the filling material is filled inside the iron matrix; or the iron matrix is a mesh skeleton structure, and the filling material is filled in meshes of the mesh skeleton structure; or the iron matrix is a hollow space skeleton structure, and the filling material is filled inside the hollow space of the iron matrix; or a groove or hole is disposed on a surface of the iron matrix, and the filling material is filled in the groove or hole of the iron matrix; or the filling material is coated on the surface of the iron matrix.

The shape of the iron matrix is a nail, mesh, plate, rod, cylinder, cube, or cone.

The orthopedic internal fixation implant medical instrument in the embodiments of the present invention may be a bone nail, bone plate, bone rod, or bone mesh.

The above medical devices are further illustrated by specific embodiments below.

The test methods involved in the following embodiments are as follows:

1. Determination of Weight-Average Molecular Weight of the Polylactic Acid

Detection is performed using a GPC-multi-angle laser light scattering instrument from Wyatt, USA coupled with a molecular weight test system. The test system includes liquid phase pump and sample injector, Agilent PL MIXED-C GPC column (dimension: 7.5×300 mm, 5 micron) from Agilent, USA, and multi-angle laser light scattering instrument and differential detector from Wyatt, USA. The detection conditions are as follows:

Mobile phase: tetrahydrofuran; pump flow rate: 1 mL/min; injection volume: 100 μL; laser wavelength: 663.9 nm; and test temperature: 35° C.

2. Polylactic Acid Mass

The weighed orthopedic internal fixation implant medical device is placed in a solvent that can dissolve the polylactic acid (such as ethyl acetate, chloroform, and the like), filtered after performing ultrasonic cleaning for 30 minutes, and weighed after drying the filtrate. The mass difference before and after cleaning is a mass of the polylactic acid.

3. Phase Identification of Alkaline Substances

The phase of alkaline substances is determined by using XRD to detect the orthopedic internal fixation implant medical device, and comparing the standard chromatograms of iron, hydroxyapatite, magnesium, zinc, magnesium oxide, zinc oxide, magnesium hydroxide, zinc hydroxide, magnesium carbonate, zinc carbonate, magnesium phosphate, zinc phosphate, sodium carbonate, sodium bicarbonate, calcium oxide, calcium hydroxide, calcium carbonate, and calcium phosphate.

4. Masses of Metal Elements in Alkaline Substances

After the orthopedic internal fixation implant medical device is digested with nitric acid, AAS is used to determine the concentration of magnesium ions or zinc ions or calcium ions or sodium ions in the digestion solution. The masses of the metal elements in alkaline substances in the orthopedic internal fixation implant medical device can be obtained through calculation.

5. Bending Strength

The three-point bending strength of the samples are tested adopting C43.504 universal material testing machine manufactured by MTS and according to YBT5349-2006 metal material bending mechanical property test standard.

6. Degradation Rate of Iron

The corrosion of the iron-based matrix is evaluated through the mass loss rate after the absorbable iron-based orthopedic internal fixation implant medical device is implanted into an animal. The included specific steps are as follows: the absorbable iron-based orthopedic internal fixation implant medical device with an iron-based matrix mass of M0 is implanted into the animal. At predetermined observation time points, such as 3 months, 6 months, 12 months, and the like, the device and its surrounding tissues are removed before soaked in a 1 mol/L sodium hydroxide solution, to remove the remaining degradable polyester and digest the tissues. The device is then removed from the sodium hydroxide solution and placed in a 3% tartaric acid solution for ultrasound, to remove all the corrosion products and other alkaline materials attached to the device or dissolve in a good solvent. The remaining device is removed, dried, and weighed to a mass of M1. The mass loss rate of the iron-based matrix at the observation time point is (M0−M1)/M0×100%.

It is considered that the iron-based matrix is not corroded if a time zone from the implantation time point to the observation time point in response to the mass loss rate W of the iron-based matrix at a certain observation time point is less than 5%. It is considered that the iron-based matrix is completely corroded in response to the mass loss rate W of the iron-based matrix at a certain observation time point is greater than or equal to 90%, the time zone from the implantation time point to the observation time point being a corrosion cycle of the iron-based matrix.

7. Local pH Value after Degradation

The orthopedic internal fixation implant medical device is immersed in a PBS solution (pH=7.4±0.1) for corrosion at 37° C. for 7 days before being removed, and the pH value on the surface of the device is immediately detected with a pH test paper.

### Embodiment 1

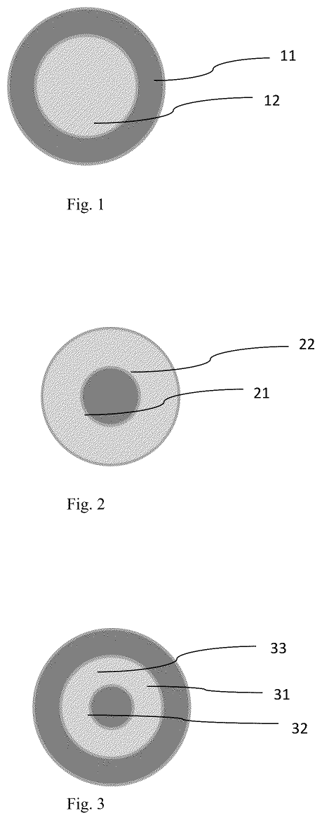

Pure iron was cast into a hollow nail with holes on the surface to obtain an iron matrix 11; pure magnesium powder and hydroxyapatite powder were dispersed in molten poly-L-lactic acid, and the mixture 12 was filled in the hollow iron matrix 11 to prepare an absorbable iron-based bone nail, the cross-section of which is shown in FIG. 1. Poly-L-lactic acid had a weight-average molecular weight of 1000 kDa, and the mass ratio of a magnesium element to poly-L-lactic acid was 0.94. The mass of hydroxyapatite was 1% of that of the whole orthopedic internal fixation implant medical device.

The initial bending strength of the bone nail was 350 MPa. The bone nail was implanted in the animal and removed after 6 months with 10% iron degradation.

The orthopedic internal fixation implant medical device was immersed in a PBS solution for a water bath at 37° C. for 7 days before being removed, and the pH value on the surface of the device was immediately detected as 7 to 8 with a pH test paper.

### Embodiment 2

Pure iron was cast into a hollow nail with holes on the surface to obtain an iron matrix; magnesium oxide particles and hydroxyapatite powder were dispersed in molten poly-L-lactic acid, and the mixture was filled in the hollow iron matrix to prepare an absorbable iron-based bone nail. Poly-L-lactic acid had a weight-average molecular weight of 1000 kDa, and the mass ratio of a magnesium element to poly-L-lactic acid was 0.008. The mass of hydroxyapatite was 1% of that of the whole orthopedic internal fixation implant medical device.

The initial bending strength of the bone nail was 350 MPa. The bone nail was implanted in the animal and removed after 6 months with 17% iron degradation.

The orthopedic internal fixation implant medical device was immersed in a PBS solution for a water bath at 37° C. for 7 days before being removed, and the pH value on the surface of the device was immediately detected as 6 to 7 with a pH test paper.

### Embodiment 3

Pure iron was cast into a nail, and the strength thereof was enhanced by ion nitriding to obtain a nail iron matrix 21; a bulk zinc alloy and hydroxyapatite powder were dispersed in molten poly-dl-lactic acid, and the mixture 22 was coated on the surface of the nail iron matrix 21 to prepare an absorbable iron-based bone nail, the cross-section of which is shown in FIG. 2. The poly-dl-lactic acid had a weight-average molecular weight of 500 kDa, and the mass ratio of a zinc element to poly-dl-lactic acid was 1.3. The mass of hydroxyapatite was 3% of that of the whole orthopedic internal fixation implant medical device.

The initial bending strength of the bone nail was 450 MPa. The bone nail was implanted in the animal and removed after 6 months with 15% iron degradation.

The orthopedic internal fixation implant medical device was immersed in a PBS solution for a water bath at 37° C. for 7 days before being removed, and the pH value on the surface of the device was immediately detected as 7 to 8 with a pH test paper.

### Embodiment 4

Pure iron was cast into a nail with grooves on the surface, and the strength thereof was enhanced by ion nitriding to obtain a nail iron matrix; magnesium hydroxide powder and hydroxyapatite powder were dispersed in molten poly-L-lactic acid, and the mixture was coated on the surface of the nail iron matrix to prepare an absorbable iron-based bone nail. The poly-L-lactic acid had a weight-average molecular weight of 500 kDa, and the mass ratio of a magnesium element to poly-L-lactic acid was 0.014. The mass of hydroxyapatite was 3% of that of the whole orthopedic internal fixation implant medical device.

The initial bending strength of the bone nail was 420 MPa. The bone nail was implanted in the animal and removed after 6 months with 18% iron degradation.

The orthopedic internal fixation implant medical device was immersed in a PBS solution for a water bath at 37° C. for 7 days before being removed, and the pH value on the surface of the device was immediately detected as 6 to 7 with a pH test paper.

### Embodiment 5

Low alloy steel was cast into a hollow nail 31, the interior of which also included an iron support rod 32 parallel to the iron nail, and the cross-section is shown in FIG. 3. The iron nail was cut into a hollow tubular structure with a laser cutter. Magnesium oxide powder and hydroxyapatite powder were dispersed in molten poly-dl-lactic acid, and the mixture 33 was filled inside an iron matrix to obtain an absorbable iron-based bone nail. The poly-di-lactic acid had a weight-average molecular weight of 100 kDa, and the mass ratio of a magnesium element to poly-dl-lactic acid was 0.05. The mass of hydroxyapatite was 10% of that of the whole orthopedic internal fixation implant medical device.

The initial bending strength of the bone nail was 380 MPa. The bone nail was implanted in the animal and removed after 6 months with 25% iron degradation.

The orthopedic internal fixation implant medical device was immersed in a PBS solution for a water bath at 37° C. for 7 days before being removed, and the pH value on the surface of the device was immediately detected as 6 to 7 with a pH test paper.

### Embodiment 6

Low alloy steel was cast into a hollow rod with holes on the surface to obtain a rod iron matrix. A small zinc rod and hydroxyapatite powder were dispersed in molten poly-dl-lactic acid, and the mixture was filled inside an iron rod to obtain an absorbable iron-based bone rod. The poly-dl-lactic acid had a weight-average molecular weight of 100 kDa, and the mass ratio of a zinc element to poly-dl-lactic acid was 3. The mass of hydroxyapatite was 10% of that of the whole orthopedic internal fixation implant medical device.

The initial bending strength of the bone rod was 480 MPa. The bone nail was implanted in the animal and removed after 6 months with 18% iron degradation.

The orthopedic internal fixation implant medical device was immersed in a PBS solution for a water bath at 37° C. for 7 days before being removed, and the pH value on the surface of the device was immediately detected as 7 to 8 with a pH test paper.

### Embodiment 7

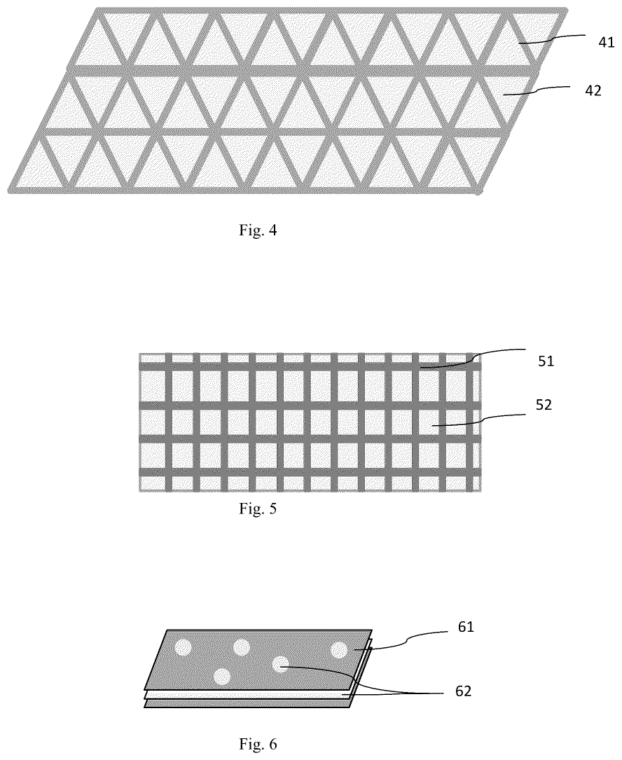

A piece of pure iron sheet was taken and cut into a mesh with a laser cutter to obtain a mesh iron matrix 41. Zinc oxide powder and hydroxyapatite powder were dispersed in molten poly-dl-lactic acid, and the mixture 42 was coated on the surface of the iron mesh and in the meshes to obtain an iron-based resorbable bone mesh, as shown in FIG. 4. The poly-dl-lactic acid had a weight-average molecular weight of 5 kDa, and the mass ratio of a zinc element to poly-dl-lactic acid was 0.55. The mass of hydroxyapatite was 6% of that of the whole orthopedic internal fixation implant medical device.

The initial bending strength of the bone mesh was 350 MPa. The bone nail was implanted in the animal and removed after 6 months with 20% iron degradation.

The orthopedic internal fixation implant medical device was immersed in a PBS solution for a water bath at 37° C. for 7 days before being removed, and the pH value on the surface of the device was immediately detected as 6 to 7 with a pH test paper.

### Embodiment 8

A piece of pure iron sheet was taken and cut into a mesh with a laser cutter to obtain a mesh iron matrix. Zinc powder and hydroxyapatite powder were dispersed in molten poly-dl-lactic acid, and the mixture was coated on the surface of the iron mesh and in the meshes to obtain an iron-based resorbable bone mesh. The poly-dl-lactic acid had a weight-average molecular weight of 5 kDa, and the mass ratio of a zinc element to poly-dl-lactic acid was 13.4. The mass of hydroxyapatite was 6% of that of the whole orthopedic internal fixation implant medical device.

The initial bending strength of the bone mesh was 500 MPa. The bone nail was implanted in the animal and removed after 6 months with 14% iron degradation.

The orthopedic internal fixation implant medical device was immersed in a PBS solution for a water bath at 37° C. for 7 days before being removed, and the pH value on the surface of the device was immediately detected as 7 to 8 with a pH test paper.

### Embodiment 9

An iron wire with a zinc layer on a hollow surface was taken and woven into an iron mesh to obtain a mesh iron matrix 51. Zinc oxide powder and hydroxyapatite powder were dispersed in molten poly-dl-lactic acid, and the mixture 52 was coated on the surface of the iron mesh and in the meshes to obtain an absorbable iron-based bone mesh, as shown in FIG. 5. The poly-dl-lactic acid had a weight-average molecular weight of 200 kDa, and the mass ratio of a zinc element to poly-dl-lactic acid was 0.03. The mass of hydroxyapatite was 7% of that of the whole orthopedic internal fixation implant medical device.

The initial bending strength of the bone mesh was 420 MPa. The bone nail was implanted in the animal and removed after 6 months with 27% iron degradation.

The orthopedic internal fixation implant medical device was immersed in a PBS solution for a water bath at 37° C. for 7 days before being removed, and the pH value on the surface of the device was immediately detected as 6 to 7 with a pH test paper.

### Embodiment 10

Pure iron was cast into a hollow iron plate 61 with holes on the surface; magnesium oxide powder and hydroxyapatite powder were dispersed in molten poly-dl-lactic acid, and the mixture 62 was filled in the middle of the iron plate 61 to obtain an absorbable iron-based bone plate, as shown in FIG. 6. The poly-dl-lactic acid had a weight-average molecular weight of 200 kDa, and the mass ratio of a magnesium element to poly-dl-lactic acid was 2.1. The mass of hydroxyapatite was 7% of that of the whole orthopedic internal fixation implant medical device.

The initial bending strength of the bone mesh was 600 MPa. The bone nail was implanted in the animal and removed after 6 months with 20% iron degradation.

The orthopedic internal fixation implant medical device was immersed in a PBS solution for a water bath at 37° C. for 7 days before being removed, and the pH value on the surface of the device was immediately detected as 7 to 8 with a pH test paper.

### Comparative Embodiment 1

A poly-L-lactic acid absorbable bone nail, with the molecular weight of poly-L-lactic acid being 500 kDa.

The initial bending strength of the bone nail was 150 MPa.

The absorbable bone nail was immersed in a PBS solution for a water bath at 37° C. for 7 days before being removed, and the pH value on the surface of the device was immediately detected as 4 to 5 with a pH test paper.

The above mentioned is only better specific implementations of the present invention, but the scope of protection of the present invention is not limited to this. Any variation or substitution that can be readily thought of by any skilled in the art within the technical scope disclosed by the present invention shall be covered by the scope of protection of the present invention. Accordingly, the protection sought herein is as set forth in the claims below.

1. An orthopedic internal fixation implant medical device, characterized by comprising an iron matrix and a filling material, the filling material comprising polylactic acid and an alkaline substance, wherein the polylactic acid has a weight-average molecular weight of M_{w} kDa, the alkaline substance comprises a metal element, wherein the mass ratio of the metal element in the alkaline substance to the polylactic acid is p, and the p and the M_{w} satisfy a formula of 2M_{w}\hat{ }−0.8≤p≤30M_{w}\hat{ }−0.5.

2. The orthopedic internal fixation implant medical device according to claim 1, characterized in that the polylactic acid has a weight-average molecular weight of 5 kDa to 1000 kDa.

3. The orthopedic internal fixation implant medical device according to claim 1, characterized in that the alkaline substance is selected from one or more of magnesium, magnesium alloy, zinc, zinc alloy, magnesium oxide, magnesium hydroxide, zinc oxide, zinc hydroxide, magnesium carbonate, zinc carbonate, magnesium phosphate, zinc phosphate, sodium carbonate, sodium bicarbonate, calcium oxide, calcium hydroxide, calcium carbonate, calcium phosphate, and hydroxyapatite.

4. The orthopedic internal fixation implant medical device according to claim 1, characterized in that the alkaline substance is a combination of hydroxyapatite and at least one of magnesium, magnesium alloy, zinc, zinc alloy, magnesium oxide, magnesium hydroxide, zinc oxide, zinc hydroxide, magnesium carbonate, zinc carbonate, magnesium phosphate, zinc phosphate, sodium carbonate, sodium bicarbonate, calcium oxide, calcium hydroxide, calcium carbonate, and calcium phosphate.

5. The orthopedic internal fixation implant medical device according to claim 1, characterized in that the alkaline substance is one or more of a powder, granule, block or rod.

6. The orthopedic internal fixation implant medical device according to claim 4, characterized in that the mass of the hydroxyapatite is 1% to 10% of that of the orthopedic internal fixation medical device.

7. The orthopedic internal fixation implant medical device according to claim 1, characterized in that the polylactic acid is poly-dl-lactic acid or poly-L-lactic acid.

8. The orthopedic internal fixation implant medical device according to claim 1, characterized in that the iron matrix is a hollow structure, and the filling material is filled inside the iron matrix; or the iron matrix is a mesh skeleton structure, and the filling material is filled in meshes of the mesh skeleton structure; or the iron matrix is a hollow space skeleton structure, and the filling material is filled inside the hollow space of the iron matrix; or a groove or hole is disposed on a surface of the iron matrix, and the filling material is filled in the groove or hole of the iron matrix; or the filling material is coated on the surface of the iron matrix.

9. The orthopedic internal fixation implant medical device according to claim 1, characterized in that the orthopedic internal fixation implant medical device is a bone nail, bone plate, bone rod, or bone mesh.

10. The orthopedic internal fixation implant medical device according to claim 1, characterized in that the iron matrix is pure iron, low alloy steel, or iron-based alloy with a carbon content of not more than 2.5 wt. %.
